(19) United States
(12) Patent Application Publication
(10) Pub. No.: US 2012/0018687 A1
(43) Pub. Date: Jan. 26, 2012

(54) Process for the preparation of easily dispersible Violet Pigment

(75) Inventors: Kisuck Jung, Pusan (KR); Sangmin Han, Ulsan (KR); Dongyoon Kim, Gyeongsangnam-do (KR); Hyunsuk Jeong, Pusan (KR); Hyunsu Lee, Ulsan (KR)

(73) Assignee: SOLVAY SA, Brussels (BE)

(21) Appl. No.: 13/260,599

(22) PCT Filed: Mar. 26, 2010
(86) PCT No.: PCT/EP2010/054020

§ 371 Date: Sep. 27, 2011

(30) Foreign Application Priority Data
Mar. 31, 2009 (EP) 09156870.9

(51) Int. Cl. G02B 5/23; C07D 498/22

(52) U.S. Cl. 252/586; 544/74

(57) ABSTRACT

The surface of a base pigment is modified so as to have a specific functional group such as sulfonic acid or its derivative, in order to increase rheological and colouristic properties and flocculation stability of dioxazine pigments (e.g., Pigment Violet 23). By modifying a pigment surface and further kneading it, dispersibility and the efficiency (brightness) of a color filter are improved without any other adverse effects.

The present application claims the benefit of the European application no. 09156870.9 filed on Mar. 31, 2009, herein incorporated by reference.

### TECHNICAL FIELD

The present disclosure relates to a pigment composition that is suitable for use in the blue pixel portion of a color filter. Further, the present disclosure relates to a process of preparing a modified dioxazine pigment having improved dispersibility in an aqueous system and excellent dispersion stability over time. The present disclosure also relates to a color filter pigment produced from the modified pigment, and a liquid crystal display device comprising said pigment.

### BACKGROUND ART

Color filters are widely used in order to display colors e.g., a liquid crystal device (LCD), which is composed of three color pixels (red, green and blue) arranged in a color matrix pattern. Methods of preparing color filters are known in the art, e.g., dyeing, printing, electrophoretic deposition, and pigment dispersion. In particular, the pigment dispersion method is widely employed wherein a composition for a color filter is obtained by dispersing pigment with a photosensitive polymer and/or a monomer. Micropixels are formed via photolithography processes, thereby creating excellent light fastness, thermal endurance and solvent resistance.

Said pigments should have characteristics to clearly display images on a LCD panel (high contrast ratio) and to display bright images (high luminance). Further, they should maintain important color characteristics, e.g., color tone, color strength, gloss and transparency. The color tone should correspond to a very specific color index. In the case of multicolor printing, the range of color palettes should be broad enough to provide a maximum number of color tones. In this regard, there has been continuous demand for a pigment that can be used in a color filter that has excellent performance with respect to color, strength, gloss and transparency, and at the same time, is able to achieve high contrast ratio and high brightness.

The pigments used in the pigment dispersion method can be selected from those commonly known in the art, and, if needed, at least two pigments can be mixed to have desirable transmission characteristics. For example, in many cases a color resist ink for blue pixels is prepared by using Pigment Blue 15:6, a blue pigment comprising copper phthalocyanine dye, together with Pigment Violet 23, a violet pigment comprising the carbazole dioxazine. Compared to the copper phthalocyanine dye, said Pigment Violet 23 has a blue-color transmitting region shifted to the shorter-wavelength. Therefore, it is widely used to improve the color purity of the blue pigment comprising copper phthalocyanine which transmits slightly green light. The color purity can be defined as absence of undesired colors in the spot produced on the screen by each RGB pigments of color filter.

By virtue of their planar chromophore structure, polycyclic and heterocyclic pigments have many properties in common: the violet pigments are virtually insoluble, even at high processing temperatures, and exhibit very good color fastness properties, including, excellent stability towards light, weathering and heat. On the other hand, they exhibit poor dispersibility and the rheology of compositions comprising them still leaves something to be desired.

The violet pigments obtained after synthesis or finishing are in many cases unsuitable for direct use. This often leads to difficulties such as incomplete dispersion and flocculation. This leads to a shortfall in the color strength and fineness of the pigment. Insufficiently dispersed pigments can also be troublesome during further processing. In particular, this leads to sedimentation problems in low-viscous systems. Furthermore, flocculation during dispersion, storage or further processing cannot be ruled out, leading to undesirable rheological modifications of the system and to surface defects, losses in color strength and gloss of the film applied.

In order to improve the properties of pigments in practical applications, it is advantageous to modify the surface of the pigment by incorporating polar functional groups to the pigment product. This leads to a distinct improvement in quality compared with the corresponding unmodified base pigment. In this respect, a number of proposals for influencing the rheological properties of pigments have been made. However, the prior art has been unable to satisfactorily address rheology, dispersibility, flocculation, flooding, gloss and color strength.

U.S. Pat. No. 5,271,759 discloses pigment compositions comprising organic pigments having a wide variety of chromophores, a sulfonated pigment derivative and a polymeric quaternary ammonium salt. The pigment compositions are said to have good rheology in surface-coatings and printing inks which enables warp-free pigmenting of the polyolefins.

U.S. Pat. No. 5,275,653 discloses pigment preparations comprising a dioxazine pigment (Pigment Violet 23) and a sulfonated dioxazine derivative as a dispersant. It is described therein that the pigment preparations have improved rheological and coloristic properties.

There are also a number of references published in the art disclosing pigment derivatives having a variety of functional groups, e.g., a dioxazine derivative having sulfonic acid groups (U.S. Pat. No. 4,400,504 and EP Pat. Publication No. 0 472 975); a dioxazine derivative having sulfonic amide groups (—SO₂—NH(CH₂)n) (JP Pat. Laid-Open No. Hei 6-228454); a sulfonated pigment having ammonium salt (U.S. Pat. Nos. 6,827,774 and 6,827,775); a pigment having carboxyl sulfonic amide groups (U.S. Pat. No. 6,918,958); a sulfonated pigment having sulfonate and/or heteroaromatic groups (U.S. Pat. No. 7,077,898).

Also disclosed is a method of treating the surface of pigment particles. U.S. Pat. No. 5,928,419 discloses a surface-treated organic pigment excellent in initial dispersibility and having long-term dispersion stability in water and in an organic solvent while being free from the coagulation of pigment particles. Such organic pigments are obtained by reacting a sulfonating agent selected from sulfamic acid or pyridine sulfur trioxide complex with an organic pigment dispersed in a solvent in which the organic pigment is insoluble or sparingly soluble. Hence, the sulfonic acid group is introduced to the surface of each particle of the organic pigment.

PCT International Publication No. WO 06011338 discloses a pigment dispersion composition which exhibits good fluidity and dispersion stability even when the pigment concentration is high, wherein the pigment dispersion is obtained by dispersing a pigment in a dispersion medium. The surface of the pigment is coated with an acid radical-containing reaction product which is produced by reacting a carbodiimide compound having at least one carbodiimide group and an acid radical-containing resin.

In preparing a dispersion of a violet pigment, a milling machine which grinds grain and other materials in the presence of milling media such as glass balls etc, or a dispersing machine in which particles are homogeneously dispersed by a shear force, etc., has been separately used since the pigment itself has a lower solubility in a solvent. For example, U.S. Pat. No. 6,451,103 and International Publication WO 05054381 disclose processes for preparing a pigment dispersion wherein dispersing machines e.g., ball mill, sand mill, air jet mill, hammer mill, etc. are employed. These machines differ from kneading apparatuses in that they employ milling media for the production of dispersions.

Among others, a valuable violet pigment must exhibit high and easy dispersibility, rheological and coloristic properties and flocculation stability. However, dioxazine pigments (like Pigment Violet 23) lack some of these properties.

The prior art documents teach modifying the surface of said pigments with surfonic acid or derivatives thereof. The pigments are modified to improve their dispersibility and the dispersion stability, thereby improving the properties of the color filter.

However, a sulfonated product wherein sulfonic acid groups are highly introduced, if used as a dispersant, causes coagulation of the obtained pigment dispersion. Further, the sulfonated product changes the color of the resultant pigment, which leads to a brighter index of the color coordination, thus increasing usage compared to the unsulfonated product. Hence, there is no effect of improving the performance of the color filter as a final product.

### BRIEF DESCRIPTION OF DRAWINGS

FIG. 1 shows TEM images of Pigment Violet 23 particles (a) before the surface-treating step, (b) after the surface-treating step and (c) after the kneading step.

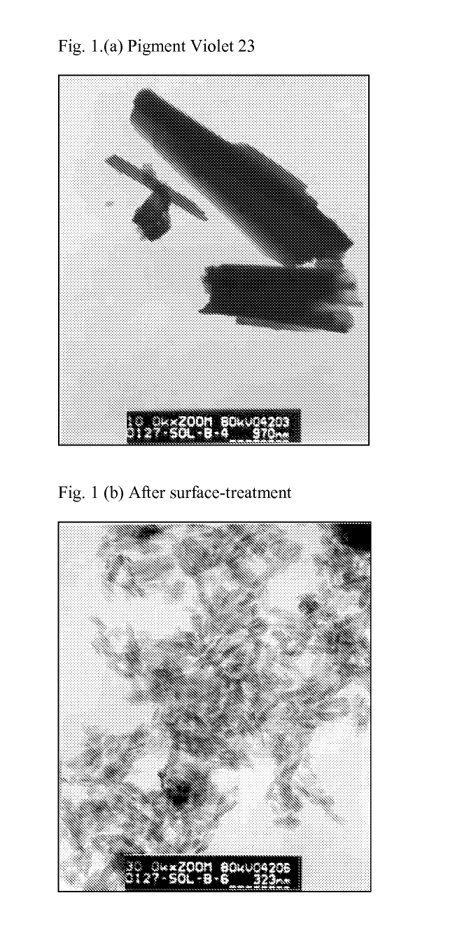

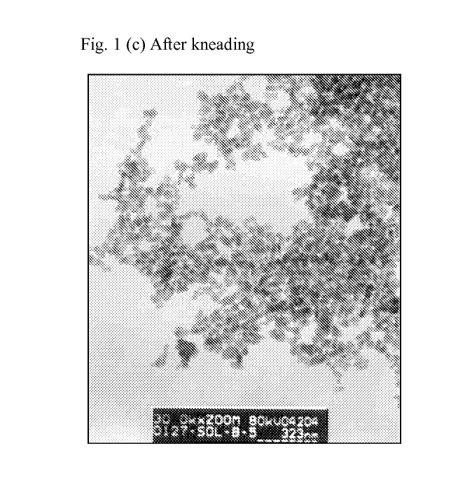

### DISCLOSURE OF THE INVENTION

Given the disadvantages of the conventional technologies, the present disclosure provides process of preparing a modified dioxazine pigment, a modified dioxazine pigment obtainable according to said process, the use of said pigment in display devices, e.g., a liquid crystal display device to form high level of pixels; a composition for use in color filters from said pigment; a cured product and a color filter obtained therefrom, and a process of preparing a pigment composition for color filters.

It was discovered modifying the surface of dioxazine pigment particles to have a polar functional group, followed by kneading, had the unexpected result of satisfying several properties important for pigments used in color filters. In particular, the modification leads to excellent contrast ratios as well as inhibiting coagulation of the obtained pigment.

The term “pigment” is defined herein to refer to a material that changes the color of light it reflects as the result of selective color absorption. The pigments display the colors because they selectively reflect and absorb certain wavelengths of light by the chemical bonds and substituents of the pigment.

The expression “functionalizing agent” for surface modification is defined herein to refer to a chemical agent to introduce chemical functional groups to the surface of particles.

In one embodiment, the dispersibility is increased while inhibiting adverse effects, e.g., coagulation. This has the effect of an excellent performance in applying a color filter of a liquid crystal element (e.g., the value of contrast ratio or “CR value”).

According to the present disclosure, when the surface of the pigment is modified to include a polar functional group on its surface and is further kneaded, surprisingly, many of the disadvantages listed above were inhibited. At the same time, rheology and contrast ratio of the resulting color filters were much improved.

The present disclosure also relates to a modified dioxazine pigment for use in color filters comprising surface-treatment of a dioxazine pigment with a functionalizing agent. Then the surface-treated pigment goes through a kneading process. In another embodiment of the present invention, the functionalizing agent is a sulfonating agent.

In another embodiment, the dioxazine pigment has an aspect ratio, which is defined herein as the height of a pigment particle with respect to the width thereof. The aspect ratio is usually determined by an image analysis of pictures taken by transmission electron microscopy (TEM) or scanning electron microscopy (SEM). The mean length (L) can be determined by several methods including measurement of the maximal Feret diameter, the length of the rectangle in which the particle can be inserted or the length L. Likewise, the mean width of the particles can be determined according to the diameter of a circle of equivalent projection area, the minimal Feret diameter, the width of the rectangle in which the particle can be inserted or the width l. Thus, the aspect ratio (L/l) corresponds to the ratio between a length (L) and the related width (l), especially the maximal Feret diameter on the minimal Feret diameter, the maximal Feret diameter on the diameter of a circle of equivalent projection area, the length of the rectangle in which the particle can be inserted on the width of the rectangle in which the particle can be inserted or the length measured directly on the width measured directly. The aspect ratio of a population of particles is defined as the mean of the aspect ratio of each particle.

In this embodiment, the aspect ratio, which is an averaged ratio of the length measured directly on the width measured directly, is usually 1:1 to 1:2. The aspect ratio can be obtained from Scanning Electron Microscope (SEM) images of the pigment particles by visually measuring the aspect ratio of each particle in the image. The ratio close to 1:1 can result in improved dispersion stability, viscosity, contrast ratio and brightness.

According to one embodiment, the substitution degree of sulfonic acid to the dioxazine pigment, which is defined herein as the number of sulfonic acid group per each pigment molecule, is in general from 0.05 to 0.5, which can be obtained from the sulphur amount measured by the Carbon/Surfur determinator. The ratio of the sulfonating agent to the pigment is usually from 10 to 40 w/w (weight/weight). By adjusting the amount of the sulfonating agent, the substitution degree of sulfonic acid to the dioxazine pigment is from 0.05 to 0.5. In specific embodiments of the present invention, the substitution degree is from 0.1 to 0.3. Further, the substitution degree is controllable by adjusting reaction conditions such as the temperature.

In another embodiment, the functionalizing agent provides to the surface of the dioxazine pigment at least one functional group selected from the group consisting of —SO₃M, —SO₂NR₁R₂ and —R₃—NR₄R₅, wherein R₁ and R₂ are independent of one another and R₁ and R₂ represent a hydrogen, alkyl, alkenyl, aryl or cycloalkyl; M represents a proton, ammonium cation or metal cation; R₃ represents a single bond, alkylene, arylene, wherein said alkylene and arylene may be substituted by at least one substituent; and R₄ and R₅ are independent of one another and represent a hydrogen, alkyl, alkenyl, aryl or cycloalkyl, or collectively form a condensed structure containing at least one of —CO—, —SO₂— and —N═N—. In some specific embodiments, the functional group is at least one selected from —SO₃M, unsubstituted or substituted PIM

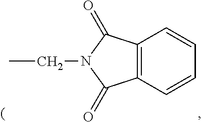

phthalimidomethylene), —SO₂NH—(CH₂)₃—N(C₄H₉)₂, and —CH₂-imidazole groups, wherein M represents a pro ton, ammonium cation or metal cation, preferably —SO₃M, especially —SO₃H.

The process of modifying the dioxazine pigment, i.e., surface-treatment, can be conducted according to methods well known in the art, e.g., sulfonating a violet pigment with concentrated sulfuric acid or fuming sulfuric acid, or chlorosulfonating the pigment with chlorosulfonic acid, followed by hydrolyzing the resulting groups. In specific embodiments, sulfonation is conducted by reacting a violet pigment with a solution of sulfuric acid having a concentration of 60-90% by weight.

The surface-treatment is conducted, whether in the presence of a solvent or not, with said sulfonating agents, e.g., concentrated sulfuric acid, fuming sulfuric acid having a concentration of from 10 to 60%, chlorosulfonic acid, sulfur trioxide and amidosulfone acid. In particular, the sulfonating agent is selected from a group consisting of chlorosulfuric acid, sulfuric acid and fuming sulfuric acid. Specifically, the solution of sulfuric acid should have a concentration of from 50 to 98% by weight. According to one embodiment, the sulfonating agent is used in a conventional manner, i.e., in an amount 5 to 50 times, specifically 5 to 20 times as much as the weight of the pigment.

As a useful solvent, acetic acid, acetic anhydride, ethyl acetate, diethyl ether, carbon tetrachloride and acetonitrile can be used in an amount 1 to 100 times, specifically 2 to 50 times, more specifically 3 to 20 times as much as the weight of the pigment.

In another embodiment, at least a portion of the functional groups can be incorporated to the surface of the dioxazine pigment by introducing a substituent such as —SO₂Cl, —SO₃H, —COCl, —CH₂Cl, etc., and reacting an amine component which reacts with the substituent such as 3-(dibutylamino)propylamine, phthalimide, etc., to form said functional group. For example, the modified dioxazine pigment can be prepared from Pigment Violet 23 (PV23), as in Scheme 1 below. In another embodiment, the functional groups are incorporated by reacting the dioxazine pigment with formaldehyde and phthalimide in the presence of acid (see Scheme 2).

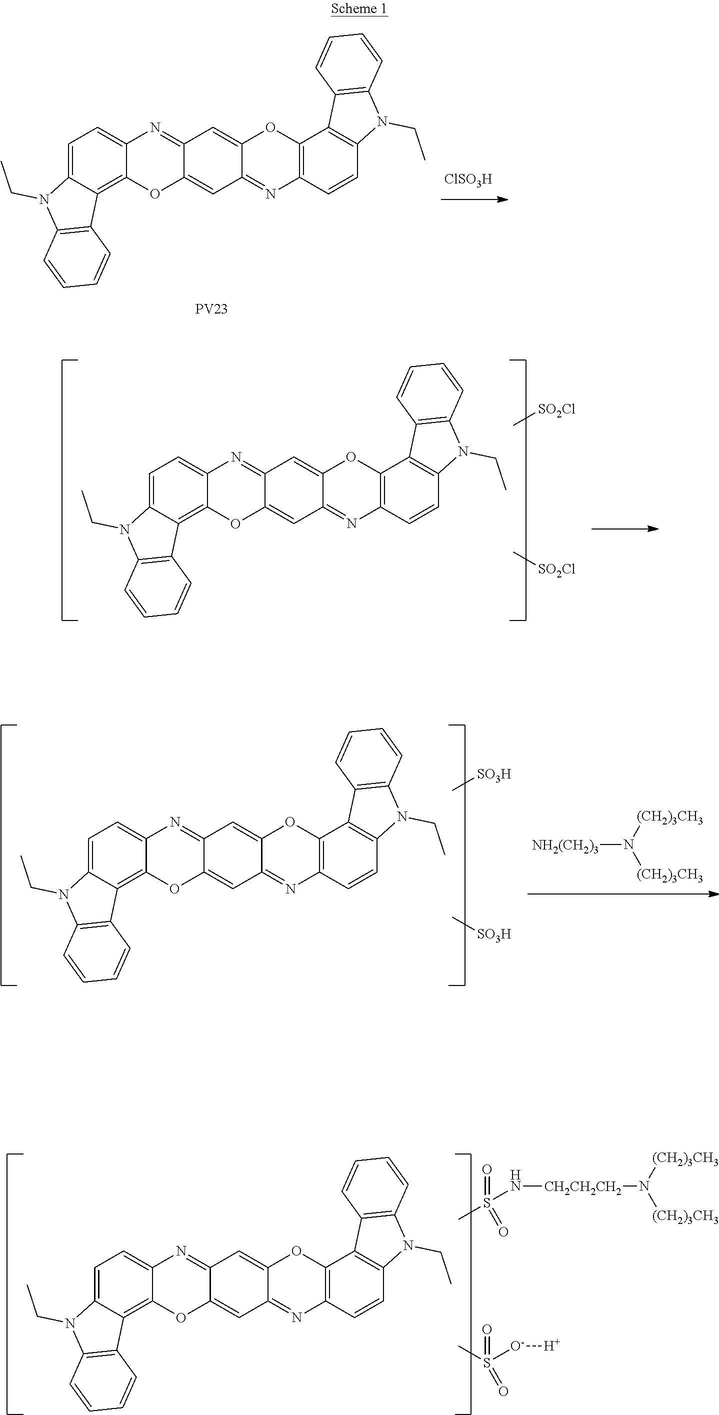

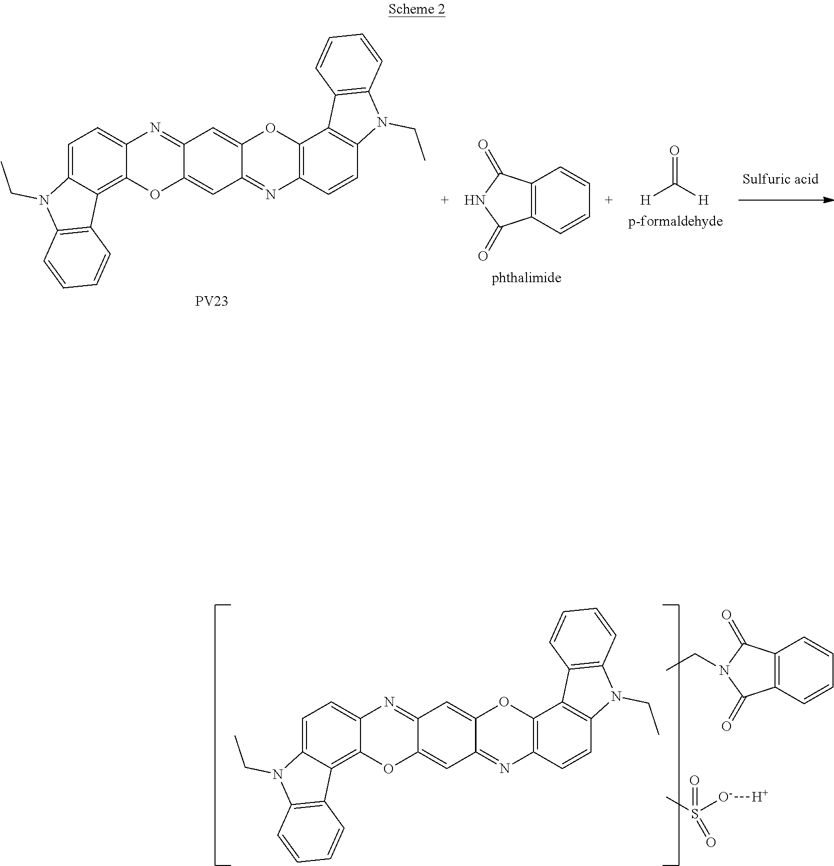

The surface-treatment is conducted at a temperature of 0 to 100° C., specifically 30 to 80° C. for at least 0.1 hour, more specifically 0.5 hours. The process is usually conducted for 10 hours or less, specifically 5 hours or less.

According to one embodiment, the dioxazine pigment includes a dioxazine skeleton represented by Formula I.

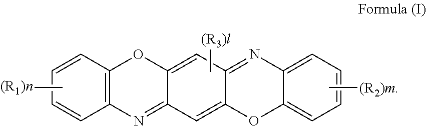

wherein:
R₁, R₂ and R₃ are same or different at each occurrence and independently selected from the group consisting of halogen, —NO₂, —CN, a straight or branched C₁₋₂₀ alkyl, a C₃₋₂₀ cyclic alkyl, a straight or branched C₁₋₂₀ alkoxy, a C₁₋₂₀ dialkylamino, a C₄₋₁₄ aryl, and a C₄₋₁₄ heteroaryl which may be substituted by one or more non aromatic radicals, wherein a plurality of substituents R₁, R₂ and R₃ may in turn together form a further mono- or polycyclic ring system, optionally aromatic;
l is an integer from 0 to 2; and
m and n are same or different at each occurrence and are an integer from 0 to 4.

Such violet pigments include C.I. Pigment Violet 23 and C.I. Pigment Violet 37. In another embodiment, the dioxazine pigment is C.I. Pigment Blue 80. In specific embodiments of the present invention, the dioxazine pigment is carbazole dioxazine, C.I. Pigment Violet 23 having a violet color. The C.I. Pigment Violet 23 is represented by the following formula:

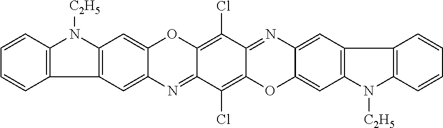

Following surface-treatment, the pigment goes through a kneading process. This improves the performance of the LCD color filter produced therefrom. Without surface-treatment, the contrast ratio (CR) value is decreased even if the dioxazine pigment has been kneaded. Furthermore, the color index of the color filter containing the pigment is changed compared to color filters containing commercially available dioxazine pigment, such effect having to be avoided. The color index is also changed compared to color filters containing unmodified dioxazine pigment or surface-treated dioxazine pigment. Surprisingly, when dioxazine pigments are prepared according to the present invention (with a surface treatment and a kneading step), the color index of the resulting color filter is unchanged compared to unmodified or surface-treated pigment, but the contrast ratio is increased.

According to one further embodiment, the process of preparing a pigment composition for a color filter comprises surface-treating a dioxazine pigment with the sulfonating agent; mixing the dioxazine pigment with a blue pigment comprising copper phthalocyanine; and kneading the mixture. In another embodiment, said blue pigment is a copper phthalocyanine exhibiting epsilon-crystallographic form.

The kneading process of the various embodiments of the present invention can be conducted by the kneading technology commonly used in the art. In one embodiment, a salt-kneading system is employed. To achieve the salt kneading process, it is possible to use a typical continuous kneading unit well known in the art, including a single kneading-screw type and a twin kneading-screw type.

The kneading step can be carried out for various durations. Generally the duration is higher than or equal to 1 hour, specifically higher than or equal to 5 hours, more specifically higher than or equal to 6 hours, and most specifically higher than or equal to 10 hours. The duration is also generally lower than or equal to 30 hours, specifically lower than or equal to 24 hours, and most specifically lower than or equal to 20 hours.

The kneading step can also be carried out at varying temperatures. Generally the temperature is higher than or equal to 0° C. and specifically higher than or equal to 10° C. and most specifically higher than or equal to 30° C. The temperature is also generally lower than or equal to 130° C., specifically lower than or equal to 100° C. and more specifically lower than or equal to 70° C.

Said kneading process can be conducted in the presence of organic liquids. The organic liquids suitable for the kneading step may include, but are not limited to, water, N-methyl-2-pyrrolidone, sulfolane, N,N-dimethyl formamide, diethylene glycol, N-methyl formamide, diacetone alcohol, glycerin, ethylene glycol, propylene glycol, polypropylene glycol, 2-butoxy ethanol, triethylene glycol, diethylene glycol monomethyl ether, triethylene glycol monomethyl ether, dipropylene glycol monomethyl ether, 1-methoxy-2-propanol, 1-ethoxy-2-propanol, ketones, quinolines and any mixture of at least two of the above. Further, in another embodiment, the liquid is diethylene glycol.

In the kneading process, the weight ratio of the organic liquid to the surface-treated pigment and the optionally added blue pigment comprising copper phthalocyanine is generally higher than or equal to 0.033 and more specifically higher than or equal to 0.050. Such weight ratio is generally lower than or equal to 1.0 and more specifically lower than or equal to 0.666.

In the kneading step, according to one embodiment, the inorganic salt suitable for the salt kneading process may include, but is not limited to, aluminum sulfate, sodium sulfate, calcium chloride, potassium chloride or sodium chloride. Specifically the sodium chloride may, if desired and available, contain water of crystallization. The weight ratio of salts to the surface-treated pigment and the optionally added blue pigment comprising copper phthalocyanine by weight ratio is generally higher than or equal to 1 (salts):0.067 (the sulface-treated pigment+the blue pigment) and specifically higher than or equal to 1:0.1. Such weight ratio is generally lower than or equal to 1:1.0 and more specifically lower than or equal to 1:0.2.

The inorganic salt also has an average particle size, which is generally higher than or equal to 0.3 μm. Such average particle size is generally lower than or equal to 200 μm, specifically lower than or equal to 50 μm. The inorganic salt is generally soluble in water, specifically to the extent of at least 10 g/100 ml of water. The average particle size of the inorganic salt may be measured using a particle size analyzer (NANOPHOX, Sympatec).

The rotary speed of the salt kneading system should be adjusted, in such a way that the kneaded composition is moved homogeneously under a uniform shear. During the salt kneading, it is possible to maintain the rotary speed in a range from 30 to 150 rpm, and more specifically from 50 to 120 rpm.

Any known blue pigment in the art comprising ε copper phthalocyanine, such as C.I. Pigment Blue 15:6, a commercial product, can be used. The primary average particle size of an ε form copper phthalocyanine pigment can be determined by selecting at least 50 first particles forming an agglomerate from the image of a Transmission Electron Microscope (TEM) and calculating the average long diameter of the particles. The diameter can vary. However, it is generally less than 40 nm, and specifically less than 30 nm. Epsilon crystal form copper phthalocyanines with smaller particle sizes improve the contrast of a color filter. Therefore, they can be effectively used as a blue filter for LCD devices. Any shape of ε form copper phthalocyanine pigment can be used. However, in some embodiments, a needle-like shape is disfavored and thus an aspect ratio is 1 or more but less than 5.

In one embodiment, copper phthalocyanine having ε crystal form is obtained by heating copper phthalocyanine particles comprising over 50 wt % of particles exhibiting ε crystal form in liquid at a temperature of 50° C. or above and optionally milling in the presence of beads. In another embodiment, copper phthalocyanine having ε crystal form is obtained by kneading copper phthalocyanine particles having over 50 wt % of particles exhibiting ε crystal form in the presence of at least one liquid and at least one inorganic salt. Specifically, the kneading is conducted under the temperature condition in which at least two points where a differential value of temperature relative to time (ΔT/Δt) is 0 exist.

The amount of copper phthalocyanine blue pigment is at least 50 wt % of the whole pigment composition, specifically at least 60 wt %, more specifically at least 70 wt %, and most specifically at least 75 wt %. The amount of copper phthalocyanine blue pigment is generally less than 100 wt %, specifically less than 99 wt %, and most specifically less than 97 wt %. The amount of the surface-treated dioxazine pigment is typically less than 50 wt %, specifically less than 40 wt %, more specifically less than 30 wt %, and most specifically less than 25 wt % The amount of the surface-treated dioxazine pigment is generally at least 1 wt %, preferably at least 2 wt %, most preferably at least 3 wt %.

Further, in order to increase properties such as dispersibility, particles of copper phthalocyanine substituted with at least one functional group selected from the group consisting of —SO₃M, —SO₂NR¹R² and —R³—NR⁴R⁵ (wherein R¹ and R² are independent of one another and represent a hydrogen, alkyl, alkenyl, aryl or cycloalkyl; M represents a proton, ammonium cation or metal cation; R³ represents a single bond, alkylene, or an arylene, wherein said alkylene and arylene may be substituted by at least one substituent; R⁴ and R⁵ are independent of one another and represents a hydrogen, alkyl, alkenyl, aryl or cycloalkyl, or collectively form a condensed structure containing at least one of —CO—, —SO₂— and —N═N—) to a phthalocyanine blue pigment, may be added.

More specifically, the copper phthalocyanine derivative particles used as a dispersion aid can be substituted by a functional group of SO₃H, —SO₂NHR¹ or

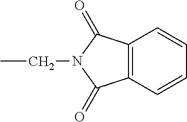

(R¹ is hydrogen, alkyl, alkenyl, aryl or cycloalkyl).

Generally, the amount of the dispersion aid is 2 to 20 wt % of the blue pigment, specifically 4 to 15 wt %, and more specifically 6 to 12 wt %. The dispersion aid may be commonly known materials in the art, or can be produced by a general process known in the art.

The pigment composition can be used solely as a pigment for the blue pixel portion of a color filter. But, if necessary, it can also be used together with different types of blue organic pigments. In the case of using different types of blue organic pigments together with the pigment composition described herein, it is desirable to use the pigment composition as a main component since it leads to better contrast ratio and brightness.

In another embodiment, dioxazine derivatives which are surface-treated by a sulfonating agent and then kneaded, are used in color filters of display devices, especially LCD devices.

The pigment compound described herein can be used for forming the blue portion of the color filter by using common methods known in the art. In preparing said blue pixel portion using the pigment, a pigment dispersion method is commonly used. The components of the pigment dispersion include colorants such as the blue and violet pigments, a binder and a polymer-based dispersion aid, pigment dispersant and liquid medium, as described in U.S. Pat. No. 7,368,148 which is incorporated herein by reference in its entirety.

Specifically, a solvent such as butanol, a dispersant and a binder (including polymeric or resin material) are mixed. Then, the modified dioxazine pigment of the present invention, a pigment comprising copper phthalocyanine exhibiting epsilon crystallographic type and the derivatives of copper phthalocyanine are added while stirring the mixture. Thereafter, the resultant mixture is subject to milling for finely-dividing the particles of the components. In some embodiments of the present invention, the milling can be conducted in at least two steps utilizing milling beads having a different size. In specific embodiments, the milling is first conducted in the presence of large beads and then in the presence of small beads. After the milling step, the beads are removed by an appropriate separation means such as a glass filter.

The present invention also relates to a pigment composition for preparing a color filter, comprising:

(a) 50 to 99% by weight, preferably 75 to 97% by weight of a blue pigment comprising copper phthalocyanine particles exhibiting epsilon crystallographic form, and

(b) 1 to 50% by weight, preferably 3 to 25% by weight of the modified dioxazine pigment of the present invention,

wherein the blue pigment comprises 2 to 20 wt %, preferably 4 to 15 wt %, and more preferably 6 to 12 wt % of a dispersion aid based on the weight of the blue pigment.

In an especially preferred embodiment of the present invention, the pigment composition for preparing a color filter comprises:

(a) 50 to 99% by weight, preferably 75 to 97% by weight of a blue pigment composition mainly consisting of epsilon copper phthalocyanine particles and copper phthalocyanine substituted with functional groups selected from SO₃H, SO₂NHR¹ and

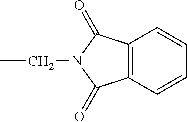

(R¹ is hydrogen, alkyl, alkenyl, aryl or cycloalkyl) in an amount of 2 to 20 wt %, preferably 4 to 15 wt %, and more preferably 6 to 12 wt % on the weight of the blue pigment composition, and

(b) 1 to 50% by weight, preferably 3 to 25% by weight of modified dioxazine pigment of the present invention, especially Pigment Violet 23 modified by sulfonic acid groups.

The present invention is further illustrated by way of examples, without limiting the scope thereto.

### EXAMPLES

To provide further elaboration, the following embodiments are provided below. However, the present invention is not limited to the working embodiments below. In addition, unless otherwise indicated, all units indicated below are based on weight.

### Example 1

### Preparation Of Pigment Violet 23 Having Sulfonic Acid Groups (—SO₃H) on the Surface Thereof

10 g of Pigment Violet 23 (PV-23) pigment were added to 100 g of 98% sulfuric acid, the mixture was stirred at 50° C. for 5 hours, and extracted in 5 kg of water. After filtration of the precipitate, the precipitate was washed until a pH of 7 was achieved. The precipitate was dried at 100° C. for at least 12 hours.

When analyzing the particles using a transmission electron microscope, it was confirmed that the original PV-23 particles were stick-shaped having an aspect ratio of 1:3.0˜5.0 but the pigment violet particles after the surface-treatment step had an aspect ratio of 1:2.0˜3.0.

### Example 2

### Preparation of Pigment Violet 23 Having Sulfamoyl Groups (—SO₂—NHR) on the Surface Thereof

40 g (0.068 mol) of PV-23 pigment were added to a mixture of 80 g of thionyl chloride and 320 g of sulfonyl chloride in a three-neck rounded flask. The resultant mixture was stirred at room temperature for 4 hours and then poured into a 4.4 kg of ice water. After filtration of the precipitate, the precipitate was washed with distilled water until a pH of 7 was achieved to obtain a wet cake.

To the obtained wet cake, 2000 g of water and 5.44 g of sodium hydroxide were added, and the mixture was stirred at 60° C. for 3 hours. Then 25 g of 3-(2-Ethylhexyloxy)propylamine were added, and the mixture was further stirred at 60° C. for 3 hours. After filtration of the precipitate, the precipitate was washed with distilled water until a pH of 7 was achieved. The precipitate was dried at 100° C. for at least 12 hours.

### Examples 3a and 3b

### Kneading of the Surface-Treated Pigment Violet 23

10 parts by weight of the surface-treated Pigment Violet 23 particles obtained respectively in Examples 1 and 2 (corresponding to examples 3a and 3b) were added to a laboratory kneader together with 10 to 30 parts by weight of diethylene glycol and 20 to 100 parts by weight of sodium chloride. The respective mixtures were kneaded at 50° C. for 20 hours. After kneading, the particles were separated by filtration and dried at a pressure of 10⁴ Pa.

After the subsequent kneading step, the particles of examples 3a and 3b were circle-shaped with an aspect ratio of 1:1.0˜2.0. By visually observing agglomeration produced during preparation of a millbase, a good dispersibility without agglomeration was confirmed for both kind of particles.

### Example 4

### Test of the Particles in Color Filters

Color filters were fabricated by using the Pigment Violet 23 particles before and after kneading (no surface treatment, kneading in the same conditions as examples 3a and 3b), after surface treatment to attach sulfonic acid groups (example 1), after surface treatment to attach sulfamoyl groups (example 2), and after surface treatment and kneading (examples 3a and 3b). The dispersion level, the contrast ratio (CR), the brightness (Br., expressed as an arbitrary unit) and the color index (CI) of the resulting color filters were checked and the results are shown in Table 1 below.

TABLE 1

| | Before Kneading | After Kneading |
|---|---|---|
| PV-23 | CI: reference value | CI: modified |
| (not surface-treated) | CR: 1,000~2,000 | CR: decreased value |
| | agglomeration existed | Br: n.m |
| PV-23 having sulfonic | CI: not modified | CI: not modified |
| acid groups | CR: 1,000~2,000 | CR: 3,000 |
| (surface-treated, ex. 1) | no agglomeration | Br: 7.5 |
| | | no agglomeration |
| PV-23 having sulfamoyl | Not measured (n.m) | CI: modified |
| groups | | CR: 3,000 |
| (surface-treated, ex. 2) | | Br: 6.0 |
| | | no agglomeration |

As can be seen from the above results, when the pigments were kneaded, the contrast ratio (CR) values were increased only for the surface-treated PV-23 pigments while the CR value was decreased for the untreated PV-23 pigment. Furthermore, the color index changed for the untreated PV-23. Accordingly, improvement in CR values can be obtained only from the pigments of the process described herein, which are subjected to partial surface-treatment and kneading.

Further, in terms of brightness and change in color index, PV-23 pigment having sulfonic acid groups exhibited an improved brightness performance while maintaining the color index, compared to PV-23 pigment having sulfamoyl groups.

### Example 5

### Kneading the Surface-Treated Pigment Violet 23 of Example 1 and Copper Phthalocyanine Having Epsilon Crystal Form (Co-Kneading)

The kneading step was conducted similarly as in Example 3a except that copper phthalocyanine particles having an average particle size of less than 30 nm and sulfonated copper phthalocyanine particles were further added to a laboratory kneader and the kneading time was reduced to 15 hours. Color filters fabricated using the mixed pigment of modified Pigment Violet 23 of example 1 and ε form copper phthalocyanine pigment particles, yielded improvements in brightness and contrast ratio by approximately 5% compared to those of Example 3a and ε form copper phthalocyanine pigment.

It will be apparent to those skilled in the art that various modifications and variations can be made to the embodiments without departing from its basic features or characteristics. Thus, the described embodiments are to be considered in all respects only as illustrative, and not restrictive. The scope of the disclosure is, therefore, indicated by the appended claims, rather than by the foregoing description. All changes within the meaning and range of equivalency of the claims are to be embraced within their scope.

Should the disclosure of any of the patents, patent applications, and publications that are incorporated herein by reference conflict with the present disclosure to the extent that it might render a term unclear, the present disclosure shall take precedence.

1. A process of preparing a modified dioxazine pigment including a dioxazine skeleton represented by Formula I, comprising:
(i) surface-treating a dioxazine pigment with a functionalizing agent, and
(ii) kneading the surface-treated dioxazine pigment. 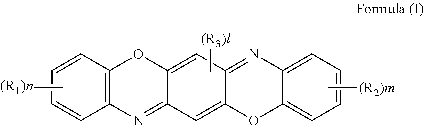
wherein:
R₁, R₂ and R₃ are same or different at each occurrence and independently selected from the group consisting of halogen, —NO₂, —CN, a straight or branched C₁₋₂₀ alkyl, a C₃₋₂₀ cyclic alkyl, a straight or branched C₁₋₂₀ alkoxy, a C₁₋₂₀ dialkylamino, a C₄₋₁₄ aryl, and a C₄₋₁₄ heteroaryl which may be substituted by one or more non aromatic radicals, wherein a plurality of substituents R₁, R₂ and R₃ may in turn together form a further mono- or polycyclic ring system, optionally aromatic;
l is an integer from 0 to 2; and
m and n are same or different at each occurrence and are an integer from 0 to 4.

2. The process according to claim 1, wherein the functionalizing agent provides to the surface of the dioxazine pigment at least one functional group selected from the group consisting of —SO₃M, —SO₂NR₁R₂ and —R₃—NR₄R₅, wherein R₁ and R₂ are independent of one another and R₁ and R₂ represent a hydrogen, alkyl, alkenyl, aryl or cycloalkyl; M represents a proton, ammonium cation or metal cation; R₃ represents a single bond, alkylene, arylene, wherein said alkylene and arylene may be substituted by at least one substituent; and R₄ and R₅ are independent of one another and represent a hydrogen, alkyl, alkenyl, aryl or cycloalkyl, or collectively form a condensed structure containing at least one of —CO—, —SO₂— and —N═N—.

3. The process according to claim 2, wherein the functionalizing agent is at least one sulfonating agent.

4. The process according to claim 2, wherein the functional group is at least one selected from the group consisting of —SO₃M, unsubstituted or substituted phthalimidomethylene (PIM), —SO₂NH—(CH₂)₃—N(C₄H₉)₂, and —CH₂-imidazole groups, wherein M represents a proton, ammonium cation, or metal cation.

5. The process according to claim 1, wherein the ratio of the functionalizing agent to the pigment is 10-40 w/w.

6. The process according to claim 1, wherein the dioxazine pigment is Pigment Violet 23.

7. The process according to claim 1, wherein surface treatment is conducted at a temperature of from 0 to 100° C.

8. The process according to claim 1, wherein kneading is conducted at a temperature of from 0 to 100° C.

9. A modified dioxazine pigment obtainable by the process according to claim 1.

10. The modified dioxazine pigment according to claim 9, having an aspect ratio of 1:1 to 1:2, wherein the substitution degree of sulfonic acid to the dioxazine pigment is from 0.05 to 0.5.

11. A method comprising preparing the modified dioxazine pigment according to claim 9 and using said modified dioxazine pigment in a color filter or in the manufacture of LCD devices.

12. A process of preparing a pigment composition for color filter, comprising:
(i) surface-treating a dioxazine pigment with a functionalizing agent,
(ii) adding a blue pigment comprising copper phthalocyanine particles exhibiting epsilon crystallographic form, and
(iii) kneading the surface-treated pigment and the blue pigment comprising copper phthalocyanine.

13. The process according to claim 12, wherein the blue pigment is copper phthalocyanine exhibiting epsilon-crystallographic form.

14. The process according to claim 12, wherein the blue pigment further comprises particles of copper phthalocyanine substituted by at least one functional group selected from the group consisting of —SO₃M, —SO₂NR₁R₂ and —R₃—NR₄R₅, wherein R₁ and R₂ are independent of one another, and R₁ and R₂ represent a hydrogen, alkyl, alkenyl, aryl or cycloalkyl; M represents a proton, ammonium cation or metal cation; R₃ represents a single bond, alkylene, arylene, wherein said alkylene and arylene may be substituted by at least one substituent; and R₄ and R₅ are independent of one another, and represent a hydrogen, alkyl, alkenyl, aryl or cycloalkyl, or collectively form a condensed structure containing at least one of —CO—, —SO₂— and —N═N—.

15. A pigment composition for preparing a color filter, comprising:
(a) from 50 to 99% by weight, preferably 75 to 97% by weight of a blue pigment comprising copper phthalocyanine particles exhibiting epsilon crystallographic form; and
(b) from 1 to 50% by weight of the modified dioxazine pigment according to claim 9,
wherein the blue pigment comprises from 2 to 20 wt % of a dispersion aid based on the weight of the blue pigment.

16. The process according to claim 2, wherein the functionalizing agent is selected from the group consisting of chlorosulfuric acid, sulfuric acid, and fuming sulfuric acid

17. A pigment composition for preparing a color filter, comprising:
(a) from 75 to 97% by weight of a blue pigment comprising copper phthalocyanine particles exhibiting epsilon crystallographic form; and
(b) from 3 to 25% by weight of the modified dioxazine pigment according to claim 9,
wherein the blue pigment comprises from 4 to 15 wt % of a dispersion aid based on the weight of the blue pigment.
